(19) United States
(12) Patent Application Publication
(10) Pub. No.: US 2017/0267563 A1
(43) Pub. Date: Sep. 21, 2017

(54) NEGATIVE HYDROGEN ION INTELLEGRNT FILTRATION SYSTEM FOR CURING CANCER, CARDIOVASCULAR DISEASE, DIABETES, HYPERTENSION, NEPHROTIC SYNDROME, GOUT, DYSAUTONOMIA AND ATOPIC DERMATITIS

(71) Applicant: Mega Sun Biomedical Corp., Taipei (TW)

(72) Inventor: CHENG-TUNG TSAN, Taipei (TW)

(21) Appl. No.: 15/075,196

(22) Filed: Mar. 20, 2016

(51) Int. Cl. C02F 9/00; A61K 41/00; C02F 1/00

(52) U.S. Cl. CPC C02F 9/00; C02F 1/005; A61K 41/0004; C02F 2101/30

(57) ABSTRACT

A negative hydrogen ion intelligent filtration system includes a housing; a filtering mechanism disposed in the housing and comprising a first filter, a second filter connected to the first filter, a third filter connected to second filter, a fourth filter connected to the third filter, a fifth filter connected to the fourth filter, a sixth filter connected to the fifth filter, a seventh filter connected to the sixth filter, an eighth filter connected to the seventh filter, a ninth filter connected to the eighth filter, a tenth filter connected to the ninth filter, a eleventh filter connected to the tenth filter and a twelfth filter connected to the eleventh filter; an inflow port connected to the first filter and extending out of the housing; an outflow port connected to the twelfth filter and extending out of the housing; and a flow rate control mechanism disposed in the housing, located between the first filter and the inflow port and connecting the first filter and the inflow port.

### FIELD OF THE INVENTION

The invention relates to a negative hydrogen ion intelligent filtration system for improving cancer, cardiovascular disease, diabetes, hypertension, nephrotic syndrome, gout, dysautonomia, atopic dermatitis, and particularly to a negative hydrogen ion intelligent filtration system which filters water to generate hydride in the water, and the filtered water provides a user the following effects: anti-oxidation, hydration, detox, turning acidic to alkaline, absorption of nutrients, immunity balance, energy generation, burning fat, strengthening metabolism, removal of free radicals and antiaging.

### BACKGROUND

Accordingly, running water often contains sand, bacteria, algae and heavy metal. If the running water is not properly processed, it may influence people's health when people drink it for a long period.

However, in addition to safety and quality of drinking water, health care effects of drinking water is also required. Therefore, a water conditioner is needed in our homestay life. Conventional water conditioners often use filters to remove sand or algae. Some simple water conditioners are even directly installed to faucet tap to filter water. Although the conventional water conditioner can remove sand and algae, the water filtered by the conventional water conditioner still lacks health benefits.

### SOME EXEMPLARY EMBODIMENTS

An object of the invention is to provide a negative hydrogen ion intelligent filtration system generating hydride (negative hydrogen ion) in water. Water filtered by the water filtration system of the invention provides a user the following effects: anti-oxidation, hydration, detox, turning acidic to alkaline, absorption of nutrients, immunity balance, energy generation, burning fat, strengthening metabolism, removal of free radicals and antiaging.

The invention provides a negative hydrogen ion intelligent filtration system for curing cancer, cardiovascular disease, diabetes, hypertension, nephrotic syndrome, gout, dysautonomia and atopic dermatitis. The negative hydrogen ion intelligent filtration system in accordance with an exemplary embodiment of the invention includes a housing; a filtering mechanism disposed in the housing and including a first filter, a second filter connected to the first filter, a third filter connected to second filter, a fourth filter connected to the third filter, a fifth filter connected to the fourth filter, a sixth filter connected to the fifth filter, a seventh filter connected to the sixth filter, an eighth filter connected to the seventh filter, a ninth filter connected to the eighth filter, a tenth filter connected to the ninth filter, a eleventh filter connected to the tenth filter and a twelfth filter connected to the eleventh filter; an inflow port connected to the first filter and extending out of the housing; an outflow port connected to the twelfth filter and extending out of the housing; and a flow rate control mechanism disposed in the housing, located between the first filter and the inflow port and connecting the first filter and the inflow port.

In another exemplary embodiment, the housing includes a base, a cover movably joined to the base, a first partition disposed in the base, a second partition disposed in the base and corresponding to the first partition and a plurality of clamps disposed on the base, the first partition and the second partition.

In yet another exemplary embodiment, the first filter, the second filter, the third filter and the fourth filter are detachably held by the clamps disposed on the base; the fifth filter, the sixth filter, the seventh filter and the eighth filter are held by the clamps disposed on the first partition; the ninth filter, the tenth filter, the eleventh filter and the twelfth filter are held by the clamps disposed on the second partition.

In another exemplary embodiment, the first filter is an antibacterial super dense fiber filter and includes antibacterial ultra-density fibers and configured to remove mud, sand, algae, microorganism and impurity.

In yet another exemplary embodiment, the second filter and the third filter are efficient water softener and configured to remove calcareous in water by ion exchange so as to soften water and remove organics, rust, oxides and toxins causing cancers.

In another exemplary embodiment, the fourth filter is a negative ion trace element filter and configured to add trace element into water so as to absorb positive ions such as calcium ion, potassium ion, magnesium ion or sodium ion and increase negative ions to remove organics and toxins in water.

In yet another exemplary embodiment, the fifth filter is a hydride generator including nano-hydrophilic catalyst and configured to generate hydride ion so as to change water electrical potential from positive potential to negative potential and generate alkaline water which has negative potential and oxidation-reduction ability to dissociate water molecules cluster.

In another exemplary embodiment, the sixth filter is a high oxygen generator including ceramics made of tourmaline and configured to release high oxygen and generate water-soluble calcium chloride to dissociate calcium oxalate.

In yet another exemplary embodiment, the seventh filter is a high efficient pionization magnetic resonator and configured to convert water molecule clusters having 12 to 14 water molecules into water molecule clusters having 6 to 8 molecules and rearrange water molecule structure so as to increase dissolved oxygen, dissolubility and penetration ability.

In another exemplary embodiment, the eighth filter including meaical stone having minerals and trace elements to generate alkaline water.

In yet another exemplary embodiment, the ninth filter is a far infrared filter to oscillate and activate water so as to generate small molecule clusters having high energy and dissolved oxygen.

In another exemplary embodiment, the tenth filter is a KDF active carbon filter having active carbon of small grain size to increase absorption area and configured to remove chlorides, disinfectants, chemical pollutants and heavy metal and inhibit bacteria and algae.

In yet another exemplary embodiment, the eleventh filter is a nano silver active carbon filter and configured to remove suspended particles, cell capsules and virus and absorb chemical substance.

In another exemplary embodiment, the twelfth filter is a molecular micronizer and configured to rearrange water molecules, convert long chain water molecules into short chain water molecules, remove pollutants and have dissolubility, penetration ability and magnetic oscillation to activate water.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 is a perspective view of a negative hydrogen ion intelligent filtration system of the invention; and

FIG. 2 is an exploded view of a negative hydrogen ion intelligent filtration system of the invention.

### DESCRIPTION OF THE PREFERRED EMBODIMENT

The following description is of the best-contemplated mode of carrying out the invention. This description is made for the purpose of illustrating the general principles of the invention and should not be taken in a limiting sense. The scope of the invention is best determined by reference to the appended claims.

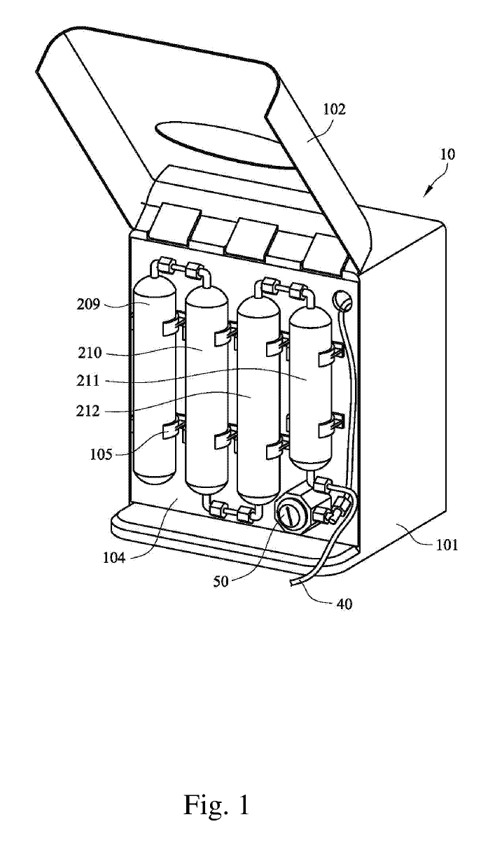

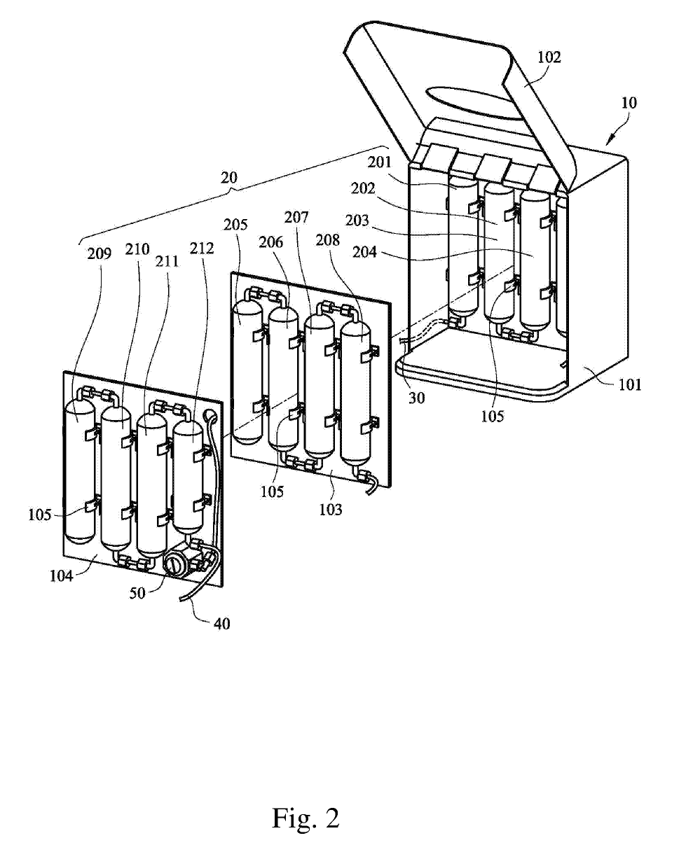

Referring to FIGS. 1 and 2, a negative hydrogen ion intelligent filtration system of the invention is applicable for improving cancer, cardiovascular disease, diabetes, hypertension, nephrotic syndrome, gout, dysautonomia and atopic dermatitis. The negative hydrogen ion intelligent filtration system of the invention includes a housing 10, a filtering mechanism 20, an inflow port 30, an outflow port 40 and a flow rate control mechanism 50.

The housing 10 includes a base 101, a cover 102 movably joined to the base 101, a first partition 103 disposed in the base 101, a second partition 104 disposed in the base 101 and corresponding to the first partition 103 and a plurality of clamps 105 disposed on the base 101, the first partition 103 and the second partition 104.

The filtering mechanism 20 is disposed in the housing 10 and includes a first filter 201, a second filter 202 connected to the first filter 201, a third filter 203 connected to second filter 202, a fourth filter 204 connected to the third filter 203, a fifth filter 205 connected to the fourth filter 204, a sixth filter 206 connected to the fifth filter 205, a seventh filter 207 connected to the sixth filter 206, an eighth filter 208 connected to the seventh filter 207, a ninth filter 209 connected to the eighth filter 208, a tenth filter 210 connected to the ninth filter 209, a eleventh filter 211 connected to the tenth filter 210 and a twelfth filter 212 connected to the eleventh filter211. The first filter 201, the second filter 202, the third filter 203 and the fourth filter 204 are detachably held by the clamps 105 disposed on the base 101; the fifth filter 205, the sixth filter 206, the seventh filter 207 and the eighth filter 208 are held by the clamps 105 disposed on the first partition 103; the ninth filter 209, the tenth filter 210, the eleventh filter 211 and the twelfth filter 212 are held by the clamps 105 disposed on the second partition 104.

The inflow port 30 is connected to the first filter 201 and extends out of the housing 10.

The outflow port 40 is connected to the twelfth filter 212 and extends out of the housing 10.

The flow rate control mechanism 50 is disposed in the housing 10, located between the first filter 201 and the inflow port 30 and connecting the first filter 201 and the inflow port 30.

Running water flows into the negative hydrogen ion intelligent filtration system through the inflow port 30. The running water flows sequentially through the first filter 201, the second filter 202, the third filter 203, the fourth filter 204, the fifth filter 205, the sixth filter 206, the seventh filter 207, the eighth filter 208, the ninth filter 209, the tenth filter 210, the eleventh filter 211 and the twelfth filter 212 in the housing 10 and flows out of the housing 10 through the outflow port 40.

The first filter 201 is an antibacterial super dense fiber filter and includes antibacterial ultra-density fibers and is configured to remove mud, sand, algae, microorganism and impurity.

The second filter 202 and the third filter 203 are efficient water softener and are configured to remove calcareous in water by ion exchange so as to soften water and also remove organics, rust, oxides and toxins causing cancers.

The fourth filter 204 is a negative ion trace element filter and configured to add trace element into water so as to absorb positive ions such as calcium ion, potassium ion, magnesium ion or sodium ion and increase negative ions to remove organics and toxins in water.

The fifth filter 205 is a hydride generator including nano-hydrophilic catalyst and configured to generate hydride ion so as to change water electrical potential from positive potential to negative potential and generate alkaline water which has negative potential and oxidation-reduction ability to dissociate water molecules cluster.

The sixth filter 206 is a high oxygen generator including ceramics made of tourmaline and configured to release high oxygen and generate water-soluble calcium chloride to dissociate calcium oxalate.

The seventh filter 207 is a high efficient pionization magnetic resonator and configured to convert water molecule clusters having 12 to 14 water molecules into water molecule clusters having 6 to 8 molecules and rearrange water molecule structure so as to increase dissolved oxygen, dissolubility and penetration ability.

The eighth filter 208 includes meaical stone having minerals and trace elements to generate alkaline water.

The ninth filter 209 is a far infrared filter to oscillate and activate water so as to generate small molecule clusters having high energy and dissolved oxygen.

The tenth filter 210 is a KDF active carbon filter having active carbon of small grain size to increase absorption area and configured to remove chlorides, disinfectants, chemical pollutants and heavy metal and inhibit bacteria and algae.

The eleventh filter 211 is a nano silver active carbon filter and configured to remove suspended particles, cell capsules and virus and absorb chemical substance.

The twelfth filter 212 is a molecular micronizer and configured to rearrange water molecules, convert long chain water molecules into short chain water molecules, remove pollutants and have dissolubility, penetration ability and magnetic oscillation to activate water.

The water filtered by the first to twelfth filters 210-212 provides a user the following effects: anti-oxidation, hydration, detox, turning acidic to alkaline, absorption of nutrients, immunity balance, energy generation, burning fat, strengthening metabolism, removal of free radicals and antiaging, and thus can improve cancer, cardiovascular disease, diabetes, hypertension, nephrotic syndrome, gout, dysautonomia and atopic dermatitis.

The first filter 201, the second filter 202, the third filter 203, the eighth filter 208, the ninth filter 209, the tenth filter 210 and the eleventh filter 211 must be replaced when 500 gallon (2200 liter) water has flowed through them. The fourth filter 204, the fifth filter 205, the sixth filter 206, the seventh filter 207 and the twelfth filter 212 can be used at least 20 years. When 500 gallon water has flowed through the first filter 201, the second filter 202, the third filter 203, the eighth filter 208, the ninth filter 209, the tenth filter 210 and the eleventh filter 211, the flow rate control mechanism 50 is closed automatically so that water cannot enter the first filer 210 thereby reminding the user to replace the first filter 201, the second filter 202, the third filter 203, the eighth filter 208, the ninth filter 209, the tenth filter 210 and the eleventh filter 211. Thus the filters are replaced when they are expired.

The negative hydrogen ion intelligent filtration system of the invention can improve cancer, cardiovascular disease, diabetes, hypertension, nephrotic syndrome, gout, dysautonomia and atopic dermatitis. Running water filtered by the negative hydrogen ion intelligent filtration system of the invention has the following effects: anti-oxidation, hydration, detox, turning acidic to alkaline, absorption of nutrients, immunity balance, energy generation, burning fat, strengthening metabolism, removal of free radicals and antiaging.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A negative hydrogen ion intelligent filtration system for curing cancer, cardiovascular disease, diabetes, hypertension, nephrotic syndrome, gout, dysautonomia and atopic dermatitis, comprising:
a housing;
a filtering mechanism disposed in the housing and comprising a first filter, a second filter connected to the first filter, a third filter connected to second filter, a fourth filter connected to the third filter, a fifth filter connected to the fourth filter, a sixth filter connected to the fifth filter, a seventh filter connected to the sixth filter, an eighth filter connected to the seventh filter, a ninth filter connected to the eighth filter, a tenth filter connected to the ninth filter, a eleventh filter connected to the tenth filter and a twelfth filter connected to the eleventh filter;
an inflow port connected to the first filter and extending out of the housing;
an outflow port connected to the twelfth filter and extending out of the housing; and
a flow rate control mechanism disposed in the housing, located between the first filter and the inflow port and connecting the first filter and the inflow port.

2. The negative hydrogen ion intelligent filtration system as claimed in claim 1, wherein the housing comprises a base, a cover movably joined to the base, a first partition disposed in the base, a second partition disposed in the base and corresponding to the first partition and a plurality of clamps disposed on the base, the first partition and the second partition.

3. The negative hydrogen ion intelligent filtration system as claimed in claim 2, wherein the first filter, the second filter, the third filter and the fourth filter are detachably held by the clamps disposed on the base; the fifth filter, the sixth filter, the seventh filter and the eighth filter are held by the clamps disposed on the first partition; the ninth filter, the tenth filter, the eleventh filter and the twelfth filter are held by the clamps disposed on the second partition.

4. The negative hydrogen ion intelligent filtration system as claimed in claim 3, wherein the first filter is an antibacterial super dense fiber filter and comprises antibacterial ultra-density fibers and configured to remove mud, sand, algae, microorganism and impurity.

5. The negative hydrogen ion intelligent filtration system as claimed in claim 4, wherein the second filter and the third filter are efficient water softener and configured to remove calcareous in water by ion exchange so as to soften water and remove organics, rust, oxides and toxins causing cancers.

6. The negative hydrogen ion intelligent filtration system as claimed in claim 5, wherein the fourth filter is a negative ion trace element filter and configured to add trace element into water so as to absorb positive ions such as calcium ion, potassium ion, magnesium ion or sodium ion and increase negative ions to remove organics and toxins in water.

7. The negative hydrogen ion intelligent filtration system as claimed in claim 6, wherein the fifth filter is a hydride generator comprising nano-hydrophilic catalyst and configured to generate hydride ion so as to change water electrical potential from positive potential to negative potential and generate alkaline water which has negative potential and oxidation-reduction ability to dissociate water molecules cluster.

8. The negative hydrogen ion intelligent filtration system as claimed in claim 7, wherein the sixth filter is a high oxygen generator comprising ceramics made of tourmaline and configured to release high oxygen and generate water-soluble calcium chloride to dissociate calcium oxalate.

9. The negative hydrogen ion intelligent filtration system as claimed in claim 8, wherein the seventh filter is a high efficient pionization magnetic resonator and configured to convert water molecule clusters having 12 to 14 water molecules into water molecule clusters having 6 to 8 molecules and rearrange water molecule structure so as to increase dissolved oxygen, dissolubility and penetration ability.

10. The negative hydrogen ion intelligent filtration system as claimed in claim 9, wherein the eighth filter comprising meaical stone having minerals and trace elements to generate alkaline water.

11. The negative hydrogen ion intelligent filtration system as claimed in claim 10, wherein the ninth filter is a far infrared filter to oscillate and activate water so as to generate small molecule clusters having high energy and dissolved oxygen.

12. The negative hydrogen ion intelligent filtration system as claimed in claim 11, wherein the tenth filter is a KDF active carbon filter having active carbon of small grain size to increase absorption area and configured to remove chlorides, disinfectants, chemical pollutants and heavy metal and inhibit bacteria and algae.

13. The negative hydrogen ion intelligent filtration system as claimed in claim 12, wherein the eleventh filter is a nano silver active carbon filter and configured to remove suspended particles, cell capsules and virus and absorb chemical substance.

14. The negative hydrogen ion intelligent filtration system as claimed in claim 13, wherein the twelfth filter is a molecular micronizer and configured to rearrange water molecules, convert long chain water molecules into short chain water molecules, remove pollutants and have dissolubility, penetration ability and magnetic oscillation to activate water.
